(19) United States
(12) Patent Application Publication
(10) Pub. No.: US 2007/0081977 A1
(43) Pub. Date: Apr. 12, 2007

(54) Device for transdermal administration of active substances

(75) Inventor: Michael Horstmann, Neuwied (DE)

Correspondence Address: D. PETER HOCHBERG CO. L.P.A., 1940 EAST 6TH STREET, CLEVELAND, OH, 44114, US

(73) Assignee: LTS Lohmann Therapie-Systeme AG, Andermach (DE)

(21) Appl. No.: 10/579,809

(22) PCT Filed: Nov. 4, 2004
(86) PCT No.: PCT/EP04/12458

§ 371 Date: May 16, 2006

(30) Foreign Application Priority Data
Nov. 17, 2003 (DE) 103 53 629.9

(51) Int. Cl. A61K 48/00; A61K 39/12; A61K 39/02; A61K 9/70

(52) U.S. Cl. 424/93.2; 604/500; 424/449; 424/204.1; 424/234.1; 514/44

(57) ABSTRACT

A device for transdermal administration of active substances, comprising a backing layer and an active substance-containing reservoir connected to the backing layer. The skin-facing contact surface of the device has a plurality of microprotrusions which are suitable for penetrating the skin and which are equipped with structures that make extracting the protrusions from the skin more difficult.

### CROSS-REFERENCE TO RELATED APPLICATIONS

This application is a National Stage application of International Application No. PCT/EP2004/012458, filed on Nov. 4, 2004, which claims priority of German application number 103 53 629.9, filed on Nov. 17, 2003.

### BACKGROUND OF THE INVENTION

1. Field of the Invention

The present invention relates to devices enabling the transdermal administration of active substances. The present invention further relates to the use of such devices for the transdermal administration of active substances or auxiliary agents.

2. Description of the Prior Art

Transdermal therapeutic systems have meanwhile become widely used as administration forms for the treatment of numerous diseases. With this technology, especially the active substances nicotine, nitroglycerin, scopolamine and estradiol, but also many more recent active substances, can be brought to action in a controlled and temporally protracted manner. It is particularly advantageous in this connection that in the case of a large number of active substances it is possible to largely suppress the physiological first-pass effect, which always occurs in the case of oral administration, when this administration form is used. This ultimately enables a larger proportion of the active substance to reach its site of action.

Usually, transdermal therapeutic systems (TTSs) are configured such that they comprise a pressure-sensitive adhesive active substance reservoir, or that in any case at least one pressure-sensitive adhesive layer is present, for attachment of the system to the skin. The adhesive bond between the system and the skin is brought about by the self-adhesive properties of the polymers used or by suitable mixtures of polymers and auxiliary agents.

The pressure-sensitive adhesive attachment on the skin, which is due exclusively to the phenomenon of surface tension, is not always reliable, however. For this reason, problems with respect to the anchoring of TTSs are observed rather frequently. Insufficient anchorage affects above all the maximum application period; generally, the usability of such systems on the skin is limited to a maximum of about 7 days.

With TTSs the release of active substances is generally affected by diffusion of the active substance through the polymer-containing and soft-plastic layers of the TTS, in which case the active substance reaches the surface of the skin via the skin-contact side of the TTS. Subsequently, the active substance molecules initially diffuse through the outermost skin layer (Stratum corneum) and then reach the deeper regions of the epidermis, where they are transferred into the blood circulation.

However, only in the case of a very small number of active substances does the afore-described diffusive transport of active substance take place at a velocity that is sufficient for therapeutic purposes. This is due above all to the natural barrier effect of the Stratum corneum. This barrier effect particularly renders the transdermal administration of high-molecular active substances (e.g. peptides, proteins, oligonucleotides and polynucleotides) or of highly polar active substances considerably more difficult, or impossible.

There has therefore been no lack of attempts to overcome the barrier property of the Stratum corneum by employing suitable methods. This can be accomplished, for example, by employing chemical permeation enhancers (e.g. ethanol or other alcohols), voltage difference (e.g. iontophoresis), or even by mechanically modifying the Stratum corneum. To this end, according to U.S. Pat. No. 6,334,856, it is possible to use a device having a plurality of hollow microneedles that are arranged in a field. These needles have a very sharp contour, it is true, but they only penetrate several hundred micrometers deep into the skin.

Although this enables the administration of active substance-containing liquids while avoiding the barrier property of the Stratum corneum, it is in many cases not possible to provide the active substances in liquid form. The reason for this may be, for example, insufficient solubility of the active substance or insufficient stability of the active substance solution. In addition, the amount of active substance which is applied is very small (in the range of a few micrograms) because of the small volume of liquid that can be applied via these microneedles.

A device which is very similar to that described in U.S. Pat. No. 6,334,856 is described in U.S. Pat. No. 6,083,196. This device comprises a carrier film which has arranged thereon a plurality of micro-protrusions that can be used for penetrating the skin. The device is fixed on the skin by an additionally present fastening means (e.g. in the form of a superimposed patch) since the device as such does not adhere to the skin.

### SUMMARY OF THE INVENTION

It was therefore the object of the present invention to provide a device for transdermal administration of active substances which is suitable, in particular, for the administration of higher-molecular or highly polar active substances and which obviates or reduces the above-described disadvantages of the prior art. More particularly, the intention is thereby to enable the transdermal administration of active substances that are not present in liquid form, and to enable adhesion of the device to the skin even without additional self-adhesive means.

This object is, surprisingly, achieved by a device according to the present invention which possesses the general features of a transdermal therapeutic system and wherein the skin-facing contact surface has a plurality of pin-shaped or needle-shaped microprotrusions which are suitable for penetrating into the skin. These microprotrusions are provided with structures that make extracting the protrusions from the skin more difficult.

The microprotrusions of the device according to the invention are configured such that to insert the microprotrusions into the skin a smaller force is required than for the subsequent extraction from the skin.

Due to the microprotrusions penetrating the skin, the barrier of the Stratum corneum is overcome and the active substances contained in the reservoir, by circumventing said barrier, are able to reach the deeper regions of the epidermis after having diffused from the reservoir to the skin-contact side of the device. The systems according to the present invention enable application of active substances into deeper skin layers, i.e. those beneath the barrier layer of the stratum corneum.

Due to the above-mentioned structures, which make extracting the protrusions from the skin more difficult, it is possible to anchor the device at the site of application on the skin, with no additional means being required to achieve this anchorage. This is a fixation which, although being similar to an adhesive bond, is based on mechanical anchorage.

The microprotrusions are preferably pointed at their distal (i.e. skin-facing) end in order to facilitate penetration into the skin. They are preferably of a needle-shaped configuration and taper towards their distal end. If the cross-section or diameter of the microprotrusions is appropriately small, penetration of the skin is also possible if the microprotrusions are not pointed or tapered. The microprotrusions have an essentially round, elliptical, triangular, quadrangular or polygonal, or an irregular cross-section. A very narrow cross-section, with the microprotrusions approximately having the shape of a saw blade or a jagged edge, should be avoided since it is thereby not possible to achieve sufficient anchorage in the skin.

The thickness of the protrusions is preferably in the range of 5 to 100 μm, especially in the range of 10 to 50 μm. In the case of needle-shaped protrusions the thickness at the point is preferably 1 to 10 μm, and at the opposite end it is preferably 5 to 100 μm.

The suitable length of the microprotrusions is dependent on the overall thickness of the device, especially on that of the active substance reservoir, as well as on the desired penetration depth. Preferably, the microprotrusions have a length in the range of from 50 to 500 μm, especially preferably in the range of from 100 to 300 μm. In this context it is additionally preferred for the microprotrusions to protrude from the skin-facing surface of the device (e.g. the polymer matrix layer) by less than 300 μm, especially less than 200 μm.

The above-mentioned structures, which make extraction from the skin more difficult and serve to anchor the device, are arranged at the outer circumference of the protrusions. There may also be a plurality (two or more) of anchoring structures present on respective ones of the protrusions, and they may be distributed along their entire length. However, at least that region of the protrusions which protrudes from the surface of the skin-contact side of the device is provided with such structures.

The anchoring structures may be realised by providing the longitudinal contour of the microprotrusions with undercuts. A configuration of these structures in the form of barbs, which counteract extraction from the skin, is particularly preferred. Each individual microprotrusion may have one or, preferably, a plurality of such barbs.

According to a further embodiment of the invention, the microprotrusions, or at least a partial quantity thereof, are configured in a helical shape and arranged rotatably. By applying a rotating movement it is thereby possible to facilitate penetration into the skin and to effect anchorage on the skin. The rotary drive may, for example, be provided by a micromechanical apparatus, particularly by micromechanical actuators.

Generally, all of the microprotrusions of a device are essentially of the same shape; however, the invention also comprises such embodiments of the device wherein two or more groups of microprotrusions of differing configuration are present.

The number of microprotrusions is preferably 1 to 10³ per mm², especially preferably 10 to 100 per mm². The microprotrusions are fixedly connected with the back layer, or/and they are embedded and fixed in the active substance-containing reservoir of the device, which is preferably configured as a polymer matrix. As an alternative, the microprotrusions can be arranged on or in an active substance-permeable film or membrane which is laminated to the skin-facing surface of the active substance-reservoir so that the, preferably pointed, ends of the microprotrusions are directed towards the outside, i.e. towards the skin. Said membrane or film may also have pressure-sensitive adhesive properties.

Particularly if the microprotrusions are anchored on a semi-rigid supporting film which serves as a back layer, there is the additional advantage that in the case of spontaneous skin movements the microprotrusions shaped according to the barb principle are caused to penetrate even deeper into the skin because the relative movements of the skin are transmitted via the back layer or supporting film to the microprotrusions.

According to a further embodiment, the microprotrusions provided with barb-like structures may also have an internal cavity or channel, in the form of a hollow needle having an opening at the distal, skin-facing end. The cavity or channel is connected with a liquid-filled reservoir, into which the hollow needles are immersed or in which they are embedded.

As an alternative, the microprotrusions may also be made of a diffusible material enabling the diffusion of the active substances from the reservoir (i.e. the active substance matrix) through the microprotrusions into the skin, so that in this case too—as in the case of the hollow needles—a direct release of active substances into the skin is possible.

The microprotrusions may be made from biocompatible and skin-friendly materials known to those skilled in the art, especially plastics and metals.

Examples of suitable plastic materials, singly or in combination, include: acrylonitrile-styrene copolymers, polymethyl methacrylates, PVC, polytetrafluoroethylene, polyamide, polyurethane and polystyrene.

Examples of suitable metal materials include: stainless steel, titanium and titanium alloys; aluminium and aluminium alloys; alloys of cobalt, chromium and molybdenum.

The microprotrusions may be made in a manner known to those skilled in the art by injection moulding, compression moulding, thermoforming, deep-drawing, extrusion, etching techniques, etc.

The invention, however, also encompasses those embodiments where the active substance release is not affected directly via the microprotrusions but via the skin-facing surface of the active substance reservoir. In that case, the function of the microprotrusions is limited to breaking through the skin barrier and to anchoring the device in the skin.

According to another embodiment, the device has an adhesive polymer matrix on the skin side, which is preferably arranged coextensively with the plane of the microprotrusions. This measure enables an even firmer fixation of the device on the skin. In this case, the microprotrusions preferably protrude from the plane of the polymer matrix layer by, on average, less than 300 μm, especially less than 200 μm. The adhesive polymer matrix may at the same time constitute the active substance reservoir and contain one or more active substances, optionally combined with one or more auxiliary agents. As an alternative, the adhesive polymer matrix may be free of active substance, the active substance(s) being present in one or more additional layers.

Suitable as base materials for the production of the said polymer matrix, the active substance-containing reservoir or a pressure-sensitive adhesive layer are, in particular, the following polymers, either singly or in combination: poly(meth)acrylates, polyisobutylenes, polyterpenes, ethylene-vinyl acetate copolymers, synthetic rubbers, styrene-isoprene-styrene block copolymers, styrene-butadiene-styrene block copolymers, hot-melt adhesives, mixtures of rubbers and resins, silicone pressure-sensitive adhesives, polyvinyl acetate, polyvinyl pyrrolidone, polyvinyl alcohols, polyethylene glycols, cellulose derivatives (e.g. hydroxypropylmethyl cellulose). Pressure-sensitive adhesive formulations based on the aforementioned polymers are in principle known to those skilled in the art.

The polymer matrix, respectively the active substance reservoir, may furthermore contain one or more known auxiliary agents, especially from the group of the softeners, emulsifiers, permeation enhancers, tackifiers, solubilisers, stabilisers, fillers and carrier substances.

The polymer matrix preferably has a polymer content of 10 to 90%-wt., especially 30 to 70%-wt.; the content of auxiliary agents is preferably between 0.1 and 30%-wt., especially between 1 and 10%-wt. The active substance content is preferably in the range of from 0.1 to 20%-wt., especially from 0.5 to 10%-wt. The weight percentage of the microprotrusions is not taken into account in the calculation.

Suitable as back layers or supporting layers are, above all, polyester films which have a particularly high strength, such as, for example, polyethylene terephthalate and polybutylene terephthalate, and in addition other skin-friendly plastic materials, such as, for example, polyvinylchloride, ethylene vinyl acetate, vinyl acetate, polyethylene, polypropylene and cellulose derivatives.

The skin-facing surface of the devices according to the invention is preferably covered with a detachable protective film, which is peeled away prior to application. The same material may be used for the detachable protective film as for the back layer, provided that the layer is subjected to a suitable surface treatment, e.g. siliconisation or fluorosiliconisation. However, other detachable protective layers, such as polytetrafluoroethylene-treated paper, cellophane, polyvinylchloride or the like, may be used as well.

The devices according to the present invention are advantageously suitable for transdermal administration of pharmaceutical active substances or vaccines for therapeutic or prophylactic treatment, including also for the purpose of immunisation, of humans or animals. They are particularly suitable for administering higher-molecular or highly polar active substances at effective oral dosages (humans) of less than 10 mg/day.

The devices according to the present invention are characterized on the one hand by a safe and long-lasting adhesion to the skin; on the other hand they enable the transdermal administration of active substances that would otherwise not be suitable for transdermal administration.

These devices therefore preferably contain one or more active substances selected from the groups of the peptides (especially peptide hormones, such as, e.g., insulin, oxytocin, vasopressin; growth factors, immunomodulators, antibiotics), proteins (e.g. enzymes, interferons, interleukins, antibodies), highly active genetically engineered active substances; oligonucleotides (e.g. antisense oligonucleotides) and polynucleotides (e.g. plasmids), or/and they contain one or more active vaccines, preferably selected from the group comprising live bacteria, killed bacteria, attenuated or genetically modified viruses, inactivated viruses, bacterial toxoids, proteins, glycoproteins, genetically engineered antigens, as well as oligonucleotides and polynucleotides.

In addition, the devices according to the present invention may also be used to administer other pharmaceutical active substances. Active substances suitable in this context are, in particular, those from the following groups: agents lowering or regulating the blood pressure; cardioactive agents (especially beta-blockers and anti-arrhythmic agents); analgesics; steroid hormones; anaesthetics; appetite depressants; anti-allergics, antihistaminics; anti-arteriosclerotic active agents; anti-arthritic/anti-rheumatic agents; antibiotics; anticholinergics; anticonvulsives; antidepressants; antidiabetic agents; antidiarrhoeal agents; antidiuretics; anti-estrogens; antimycotics/fungicide active agents, active agents against gout; lipid-lowering agents; hormones; non-steroidal antiphlogistics; anti-migraine agents; agents against nausea; antineoplastic agents; anti-Parkinson agents; antipsychotic agents; antispastic/antispasmodic agents; antithrombotics; antiviral agents; anxiolytics; bronchodilators; calcium channel blockers; cholinergics; cholinesterase inhibitors; CNS stimulants; dopamine receptor agonists; immunomodulators, immunosuppressive agents; ion-exchange resins; monoamine oxidase inhibitors; sedatives/hypnotics; thrombolytics; vasodilators; vitamins.

The invention will now be explained with reference to the schematic representations of FIGS. 1 to 3. The embodiments shown therein are merely by way of example.

### BRIEF DESCRIPTION OF THE DRAWINGS

FIG. 1 is a cross-sectional view of a first embodiment of a device according to the present invention.

FIG. 2 is a cross-sectional view of a second embodiment of a device according to the present invention.

FIG. 3 is a cross-sectional view of a drive apparatus for use with either embodiment of the present invention.

### DETAILED DESCRIPTION OF THE PRESENT INVENTION

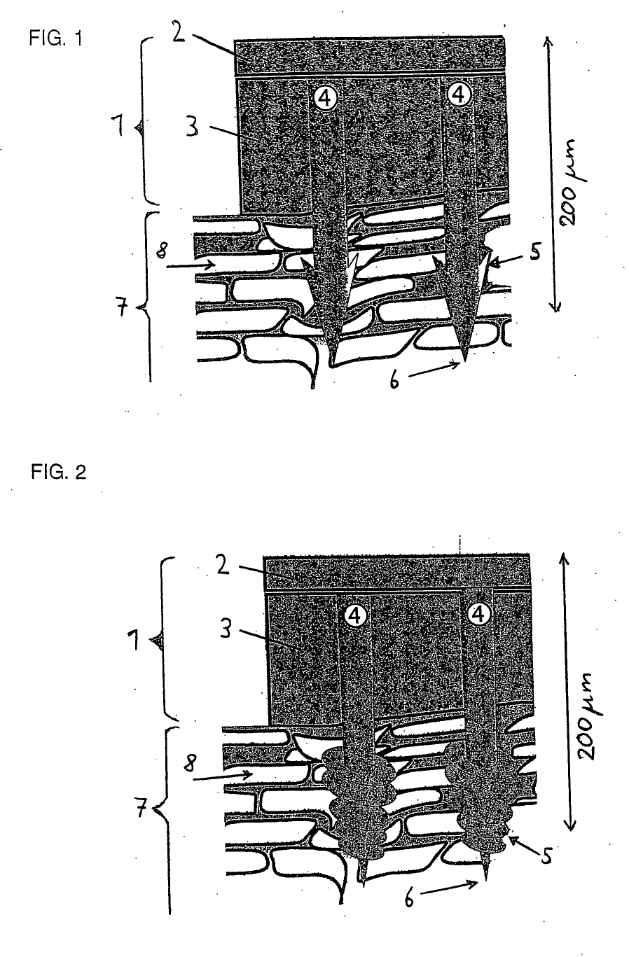

FIGS. 1 and 2 each show a longitudinal section of a device 1 according to the invention for transdermal administration of active substance, which device is in the state of having been applied to the skin 7. The comeocytes embedded in the lipid matrix are shown generally at numeral 8.

FIG. 1 shows a device 1 having a back layer or supporting film 2 and an active substance-containing polymer matrix 3. In this matrix, there is a plurality of microprotrusions 4, two of which are shown.

At the distal end of the substantially cylindrical shank, the microprotrusions 4 have a point 6 with which they penetrate into the skin 7. The opposite end of the microprotrusions 4 is connected with the backing layer 2, so that the microprotrusions 4 are arranged approximately perpendicular to the plane of the back layer 2.

The microprotrusions have barb-shaped anchoring structures 5. The cross-section of the microprotrusions is round; the same is true of the anchoring structures 5.

FIG. 2 shows a modification of the device depicted in FIG. 1, wherein the anchoring structures 5 of the microprotrusions 4 are of a helical configuration. The microprotrusions are rotatingly guided and are made to rotate by a micromechanical drive apparatus (not shown). The reference numbers otherwise have the same meaning as in FIG. 1.

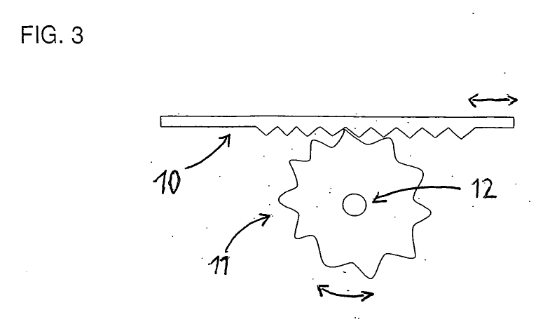

FIG. 3 shows (in section) an example of a micromechanical actuator that may be employed as a drive apparatus in a device according to the invention, e.g. in a device according to FIG. 2.

By moving a micro-rack 10 in one of the directions of the arrow, the micro-gear 11 is driven. The axle 12 of the gear may be provided in the distal (skin-facing) region with a helical anchoring structure, as shown in FIG. 2.

By employing a plurality of gear racks 10 and gears 11 it is possible to affect a unidirectional drive of a plurality of helical microprotrusions 4, 5. In this case, it is furthermore possible to jointly drive two or more gears 11 by a respective single rack.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. A device for transdermal administration of active substances, comprising a back layer and an active substance-containing reservoir connected to said back layer, wherein said device has a skin-facing contact surface on a side facing the skin, said skin-facing contact surface comprising a plurality of needle-shaped microprotrusions for penetrating into the skin and having a longitudinal contour having at least one undercut for rendering extraction of the plurality of protrusions from the skin more difficult and for fixing the device on the skin.

2. The device according to claim 1, wherein said plurality of microprotrusions are configured as a plurality of barbs, and wherein each of said microprotrusions comprises at least one barb.

3. The device according to claim 1, wherein said plurality of microprotrusions are helically configured and rotatably arranged, to facilitate penetration into the skin and to affect anchorage in the skin by applying a rotating movement onto said plurality of microprotrusions.

4. The device according to claim 3, wherein micromechanical actuators cause the rotating movement of said rotatable microprotrusions.

5. The device according to claim 1, wherein at least two of said microprotrusions are fixed in the active substance-containing reservoir.

6. The device according to claim 1 wherein at least two of said microprotrusions are connected with the back layer.

7. The device according to claim 1, wherein at least two of said microprotrusions are hollow needles.

8. The device according to claim 1, wherein said device comprises an adhesive polymer matrix on the skin side arranged coextensive with the plane of the microprotrusions.

9. The device according to claim 8, wherein the microprotrusions protrude from the plane of the polymer matrix layer by less than 300 μm on average.

10. The device according to claim 9, wherein the adhesive polymer matrix also constitutes the active substance reservoir and contains at least one active substance, optionally in combination with at least one auxiliary agent.

11. The device according to claim 1, wherein said device contains at least one active substance which is selected from the group consisting of peptides, proteins, oligonucleotides and polynucleotides.

12. The device according to claim 1, wherein said device contains at least one vaccine selected from the group consisting of bacteria, viruses, bacterial toxoids, oligonucleotides, polynucleotides and genetically engineered antigens.

13. A use of a device according to claim 1 for transdermal administration of active substances or vaccines to a human or animal body.

14. The use according to claim 13, wherein the active substances or vaccines are selected from the group consisting of peptides, proteins, oligonucleotides, polynucleotides, bacteria, viruses, inactivated viruses, bacterial toxoids, oligonucleotides, polynucleotides and genetically engineered antigens.
